(19) United States
(12) Patent Application Publication
(10) Pub. No.: US 2007/0244199 A1
(43) Pub. Date: Oct. 18, 2007

(54) ANTI-MYCOBACTERIAL FORMULATION

(75) Inventors: Nicole M. Parrish, Ellicott City, MD (US); Albert Owens, Bel Air, MD (US); James D. Dick, Upperco, MD (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: FASgen Inc., Baltimore, MD (US)

(21) Appl. No.: 11/672,909

(22) Filed: Feb. 8, 2007

Related U.S. Application Data
(60) Provisional application No. 60/771,935, filed on Feb. 8, 2006.

(51) Int. Cl. A61K 31/165; C07C 233/05; C12N 1/20; C12N 1/38

(52) U.S. Cl. 514/625; 435/243; 435/253.1; 564/192

(57) ABSTRACT

The invention provides anti-microbial compositions, including compositions with inhibitory activity against mycobacteria. The invention further provides methods for treating microbial infections, including the treatment of mycobacterial infection and diseases such as tuberculosis.

### RELATED APPLICATIONS

This application claims benefit of priority to U.S. Provisional Patent Application 60/771,935, filed Feb. 8, 2006, which is hereby incorporated by reference in its entirety as if fully set forth.

### STATEMENT AS TO RIGHTS TO INVENTIONS MADE UNDER FEDERALLY SPONSORED RESEARCH AND DEVELOPMENT

The present invention was supported in part with funding provided by SBIR Phase I Grant number-1 R43 AI052680-01 and Grant number-1 R43 AI064966-01 as well as Drug Development for MDRTB Grant number-UO1 AI054842-03. The federal government may have certain rights to this invention.

### FIELD OF THE INVENTION

This invention relates to anti-microbial compositions, including, for example, compositions with inhibitory activity against mycobacteria. The invention further relates to the treatment of microbial infections, including, for example, the treatment of mycobacterial infection.

### BACKGROUND OF THE INVENTION

Microbial infections, and increasing drug resistance among microbes, remain a major public health issue in the United States and around the world. Tuberculosis (TB), for example, is a microbial infection causing morbidity and mortality in the global population. It is believed that approximately 1.86 billion people, or 32% of the world's population, are infected with Mycobacterium tuberculosis.

While this level of infection results in millions of new active cases of TB and millions of deaths each year, the majority of infected individuals carry a latent infection. The occurrence of drug resistance and multi-drug resistance (MDR) in TB is believed to result in part from the need for an extended treatment course of six to nine months. Lapses during the treatment period allows for the development of drug resistant mycobacteria. The slow growth of TB and its latency are believed to contribute to the occurrence of drug resistance.

The increasing incidence of drug resistance has led to the use of cocktails to treat TB as well as an interest in identifying of new mycobacterial targets and drug development. Drugs under development including long-acting rifamycins, fluoroquinolones, oxazolidinones, andnitroimidazoles. In late 2004, a new diarylquinoline (DARQ) compound, called R207910, was introduced as a possible alternative to a cocktail containing rifampin, isoniazid and pyrazinamide, which is prescribed for six to nine months.

Other drugs targeting energy metabolism and fatty acid metabolism in mycobacteria have also been described. WO 04/004712 is directed to decreasing ATP levels, while U.S. Pat. Nos. 6,713,654 and 5,614,551 relate to the inhibition of fatty acid synthesis.

Citation of the above documents is not intended as an admission that any of the foregoing is pertinent prior art. All statements as to the date or representation as to the contents of these documents is based on the available information and does not constitute any admission as to the correctness of the dates or contents of these documents.

### BRIEF SUMMARY OF THE INVENTION

The invention provides a composition of matter having at least anti-mycobacterial activity. A composition of the invention contains at least three agents. The first agent is n-octanesulphonylacetamide (OSA) or the sulfoxide form thereof. The second agent is n-nonanesulphonylacetamide (NSA) or the sulfoxide form thereof, and the third agent is n-decanesulphonylacetamide (DSA) or the sulfoxide form thereof.

The agents OSA, NSA, and DSA are represented by the formula NH₂—CO—CH₂—SO₂—(CH₂)ₙ—CH₃, wherein n is 7, 8, and 9, respectively. The sulfoxide form of each agent is the modification of the —SO₂—, sulfone moiety in each agent.

As evident by even a simple review, the three agents are similar in structure. They each contain an acetamide moiety on one end, followed by a single —CH₂— linker moiety attached to a sulfone or sulfoxide, which is in turn attached to an acyl chain. It is the length of the acyl chain that differs in each of the agents. Used individually, each agent differs from the other two in terms of their anti-mycobacterial activity. Generally, the most active agent is DSA, followed by NSA, and then OSA.

The instant invention is based in part on the discovery of a surprising and unexpected level of activity when the three agents are used in combination. There was no indication from the knowledge of the individual agents that, when used in combination, they would have anti-mycobacterial potency and efficacy greater than DSA, the most potent agent, as described herein. DSA was observed to be effective when used at levels below its minimum inhibitory concentration (MIC in an in vitro assay described herein) with the inclusion of NSA and OSA, also at levels below their MICs. Therefore, the invention provides for the improvement in the effectiveness of DSA, whether at MIC or sub-MIC levels, by including the use of both NSA and OSA. Of course combinations of DSA at levels above its MIC with NSA and OSA are also provided. The “MIC” may be that of the in vitro assay or another assay used to evaluate anti-mycobacterial activity of these agents.

Thus in a first aspect, the invention provides a composition comprising octanesulphonylacetamide (OSA) or the sulfoxide form thereof, nonanesulphonylacetamide (NSA) or the sulfoxide form thereof, and decanesulphonylacetamide (DSA) or the sulfoxide form thereof, wherein OSA, NSA, and DSA are represented by the formula provided above. In some embodiments, the composition may be “sulfone-only”, in that all three agents are present in the form as represented by the formula, or “sulfoxide-only”, in that all three agents are present in their sulfoxide forms. In other embodiments, the composition may be a mixture of the sulfone and sulfoxide forms. Non-limiting examples include compositions wherein both the sulfone and sulfoxide forms of one, two or all three of the agents are present. Other non-limiting examples include those wherein any one or two of the agents are present in only their sulfone or sulfoxide forms while the other two, or one, agent(s) are present in either the sulfone or sulfoxide form only.

In another aspect, the invention provides compositions wherein the agents are present in approximately equal amounts by weight or molarity. As evident from their structures, the molecular weights of the three agents are quite similar and so amounts by weight and molarity only differ slightly. In other embodiments, and given the higher MICs for NSA and OSA relative to DSA, compositions comprising DSA and higher amounts, by weight or molarity, of NSA and OSA are also provided. Of course embodiments of the invention also include the use of NSA and OSA in amounts less than that of DSA, by weight or molarity.

A composition of the invention may be in any convenient form. Solid, liquid, and suspended formulations are all provided. In some embodiments, the invention provides for solid formulations of a composition. In some cases, a solid formulation is a dosage form, used to deliver the composition to a subject in need thereof. Non-limiting examples of solid formulations include those which deliver the composition 1) based on an amount per body mass of the subject or 2) to achieve a serum or target tissue concentration in the subject. In other embodiments, the formulation is based on both 1) and 2) above.

In other embodiments, a composition is in the form of a vesicle, including unilamellar and multilamellar vesicles. Non-limiting examples of vesicles include liposomes and micelles. The vesicle may be in a dried form suitable for rehydration to reconsititute the vesicle.

A composition of the invention may further containing additional agents. In some embodiments, the additional agent is a carrier for the three agents listed above. Non-limiting examples include an inert substance or a pharmaceutically acceptable excipient. In other embodiments, the additional agent may be a solubility enhancer. Of course the invention also provides for combinations of additional agents to be used.

In another aspect, the invention provides for a mycobacterial cell contacted with a composition as provided herein. In some embodiments, the mycobacterial cell is a pathogenic cell, which may be considered to be those capable of causing a communicable disease. Non-limiting examples of pathogenic mycobacteria include Mycobacterium tuberculosis; drug resistant, including multi-drug resistant, Mycobacterium tuberculosis; Mycobacterium bovis, Mycobacterium bovis BCG (Bacillus Calmette-Guerin), Mycobacterium kansasii, Mycobacterium avium, Mycobacterium avium intracellulare, Mycobacterium leprae, Mycobacterium ulcerans, and Mycobacterium avium subspecies paratuberculosis (PTB also known as M. paratuberculosis).

In some embodiments, a mycobacterial cell is actively growing. Non-limiting examples include cells that are in an acute infection in vivo and actively proliferating cells in vitro. In other embodiments, a mycobacterial cell is not actively growing. Non-limiting examples include a cell that is in a latent infection in vivo or that is exhibiting latent growth in vitro.

In other embodiments, the cell may be opportunistic, such as mycobacteria which do not cause disease in healthy individuals but can cause under abnormal conditions. Non-limiting examples of abnormal conditions include those where the mycobacteria are present in an abnormal location within the individual or where the individual is immunocompromised or suppressed. Opportunistic mycobacteria may be considered to be those which do not cause a communicable disease among normal individuals.

In a further aspect, the invention provides for the composition to inhibit growth or proliferation of a contacted mycobacterial cell. In some embodiments, the cell is a pathogenic mycobacterial cell as described herein. Thus the invention provides methods to inhibit growth or proliferation of a mycobacterial cell. A non-limiting example of such a method is where a mycobacterial cell is contacted with a composition of the invention. The contacted cell then is inhibited in its growth or proliferation. Non-limiting examples of such cells include pathogenic mycobacteria.

In some embodiments, the inhibition of a mycobacterial cell occurs in the context of an animal subject, including a human subject. The inhibition may be mediated by administration of a composition of the invention to the subject by any suitable means, including, but not limited to, systemic or topical application.

In a yet further aspect, the administration to a subject or individual is performed as part of a method to treat a mycobacterial infection. In some embodiments, the method is used to treat an acute or latent infection. Non-limiting examples include the treatment of infection by a pathogenic mycobacterium. And while the subject may be any animal susceptible to mycobacterial infection, the invention may be advantageously used to treat a human subject. When used to treat a latent infection, the method may be considered to be directed to the inhibition or prevention of mycobacterial reactivation.

In an additional aspect, the administration to a subject or individual is performed as part of a preventive action to reduce the chances of mycobacterial infection, both acute and latent. The invention provides methods to prevent or reduce the likelihood of infection by conditioning a subject to resist mycobacterial infection. Such a method comprises the administering of a composition to a subject or individual prior to contact with mycobacteria or an indication that the subject or individual has already been exposed or infected. These methods may also be considered methods of preventing or reducing the likelihood of mycobacterial colonization, such as on the surface of epithelial tissues.

These methods of the invention may be considered to be prophylactic in nature, which is supported by observations regarding the compositions of the invention. Without being bound by theory, and offered only to aid in the understanding of the invention, it is pointed out that because the compositions appear bactericidal to mycobacterial cells, the compositions are believed to directly result in cell death. The mechanism of such bactericidal activity is unclear, but, and again without being bound by theory, it is believed that the compositions of the invention inhibit energy metabolism in a mycobacterial cell. Based upon this belief, the invention thus includes a method of inhibiting a mycobacterial ATP synthase by contacting said synthase with a composition of the invention. The targeting of a mycobacterial ATP synthase, which has little homology with mammalian ATP synthases, would advantageously result in added specificity for the compositions to selectively target mycobacteria in a mammalian host environment.

Therefore, and in further aspects, the invention further provides methods to kill mycobacterial cells and/or inhibit or reduce energy metabolism therein. Such methods comprise the contacting of a mycobacterial cell with a composition of the invention. In some embodiments, the cell is a pathogenic mycobacterial cell.

In another aspect, the invention provides methods to treat a mycobacterial infection with reduced emergence of resistance in the treated mycobacteria. The treating of mycobacteria may be with a composition of the invention to inhibit or reduce their growth, kill the mycobacterial cells, and/or inhibit or reduce energy metabolism in the mycobacterial cells with decreased occurrence of resistance to the composition. Such methods comprise the contacting of mycobacteria with an effective amount of a composition of the invention such that the emergence of resistance to the composition is reduced, when compared to use of the individual agents of the composition. So the amount of the composition is both effective to inhibit or reduce mycobacterial growth, be lethal to the treated mycobacterial cells, and/or inhibit or reduce energy use in the mycobacterial cells while also preventing the generation of resistant mycobacteria. The methods may also be viewed as reducing or minimizing selection for resistant mycobacteria.

In yet additional aspects, the invention provides methods to prepare a composition of the invention. In some embodiments, the preparation comprises combining more than one preparation, wherein each preparation comprises at least one of DSA, NSA, and OSA, or the sulfoxide forms thereof.

The details of additional embodiments of the invention are set forth in the accompanying drawings and the description below. Other features and advantages of the invention will be apparent from the drawings and detailed description, and from the claims.

### BRIEF DESCRIPTION OF THE DRAWINGS

FIG. 1 illustrates the in vitro activity of DSA, NSA, and OSA individually at concentrations of 1.5 μg/ml or less against exposed versus unexposed (control, CT) cultures of M. bovis BCG. At these low concentrations, none of the compounds exhibited significant inhibition of BCG. However, DSA showed some activity at 1.5 μg/ml.

FIG. 2 illustrates the growth index changes in an unexposed diluted control versus cultures exposed to individual compounds at various concentrations and combinations of the three compounds. The control represents 1% of the original starting inoculum. Numbers represent the change in growth index from one day to the next during the time period used to calculate the MIC using the following criterion: when the ΔGI in the control >30, the ΔGI in the exposed cultures is calculated for the same time interval. The MIC must have a ΔGI of <30. Values >30 indicate the MIC is at a higher concentration. As shown, only when the 3 compounds are combined is the MIC achievable in the lower concentration range.

FIG. 3 illustrates the activity of combinations of DSA, NSA, and OSA using two different ratios in comparison to the unexposed control culture (NC). A, B, C, and D, represent compound ratios of 6:3:1 where A=MIC of each respective compound in combination (DSA, 1.5 μg/ml; NSA, 3.0 μg/ml; OSA, 6.25 μg/ml), B=1/2 each respective MIC, C=1/4 each respective MIC, D=1/8 each respective MIC. Concentrations listed as 1.5, 0.75, 0.38, and 0.19 are in μg/ml and each compound is combined at that concentration.

FIG. 4 illustrates a comparison of changes in the BACTEC GI on day 6 of incubation in unexposed control cultures of M. avium subspecies paratuberculosis (PTB) versus cultures exposed to the triple combination of DSA, NSA, and OSA. The MIC for these compounds individually with all strains of PTB is approximately 50.0 mg/ml. Triple combinations were done at 1/2 the MIC (25.0 μg/ml for each compound) or 1/4 the MIC (2.5 μg/ml for each compound). Results from six different strains of PTB are shown and designated by number at the bottom of the graph.

FIG. 5 depicts changes in the BACTEC growth index over time in exposed versus unexposed cultures of PTB (strain #2). As shown, DSA and NSA alone at the highest concentration show some activity against PTB, whereas, no activity is demonstrated with OSA. However, when all 3 compounds are combined at either 12.5 μg/ml or 25.0 μg/ml, significant activity is observed.

FIG. 6 depicts changes in the BACTEC growth index over time in exposed versus unexposed cultures of PTB (strain #4). As shown, DSA and NSA alone at the highest concentration show some activity against PTB, whereas, no activity is demonstrated with OSA. However, when all 3 compounds are combined at either 12.5 μg/ml or 25.0 μg/ml, significant activity is observed.

FIG. 7 depicts changes in the BACTEC growth index over time in exposed versus unexposed cultures of PTB (strain #8). As shown, DSA and NSA alone at the highest concentration show some activity against PTB, whereas, no activity is demonstrated with OSA. However, when all 3 compounds are combined at either 12.5 μg/ml or 25.0 μg/ml, significant activity is observed.

FIG. 8 depicts changes in the BACTEC growth index over time in exposed versus unexposed cultures of PTB (strain #13). As shown, DSA and NSA alone at the highest concentration show some activity against PTB, whereas, no activity is demonstrated with OSA. However, when all 3 compounds are combined at either 12.5 μg/ml or 25.0 μg/ml, significant activity is observed.

FIG. 9 depicts changes in the BACTEC growth index over time in exposed versus unexposed cultures of PTB (strain #15). As shown, DSA and NSA alone at the highest concentration show some activity against PTB, whereas, no activity is demonstrated with OSA. However, when all 3 compounds are combined at either 12.5 μg/ml or 25.0 μg/ml, significant activity is observed.

### DETAILED DESCRIPTION OF MODES OF PRACTICING THE INVENTION

The invention provides anti-mycobacterial compositions and methods for their use and preparation. A composition of the invention contains the sulfone or sulfoxide form of at least three agents. In their sulfone forms, the agents are n-octanesulphonylacetamide (OSA), n-nonanesulphonylacetamide (NSA), and n-decanesulphonylacetamide (DSA). Compositions of the invention may thus comprise one of the following eight combinations.

1) DSA sulfone, NSA sulfone, and OSA sulfone;

2) DSA sulfone, NSA sulfone, and OSA sulfoxide;

3) DSA sulfone, NSA sulfoxide, and OSA sulfone;

4) DSA sulfone, NSA sulfoxide, and OSA sulfoxide;

5) DSA sulfoxide, NSA sulfone, and OSA sulfone;

6) DSA sulfoxide, NSA sulfone, and OSA sulfoxide;

7) DSA sulfoxide, NSA sulfoxide, and OSA sulfone; and

8) DSA sulfoxide, NSA sulfoxide, and OSA sulfoxide.

The agents DSA, NSA, and OSA have been previously described (see U.S. Pat. No. 6,713,654). Each agent differs from the other two in terms of their anti-mycobacterial, with DSA being the most active in an in vitro model of mycobacterial cell growth inhibition (and bactericidal), followed by NSA, and then OSA in terms of effectiveness. DSA has been observed to be active in an in vitro sterilizing model and to kill latent (hypoxic) mycobacteria in vitro

In an in vitro model based on inhibition of M. bovis BCG growth, the minimum inhibitory concentration (MIC) of each agent was determined as described below. The MIC for DSA, NSA, and OSA are 1.5, 3.0, and 6.25 μg/ml, respectively. Similar MICs were seen with M. tuberculosis. Use of each agent in amounts less than their respective MICs, such as half of the MIC, was observed to result in significantly less inhibition of cell growth.

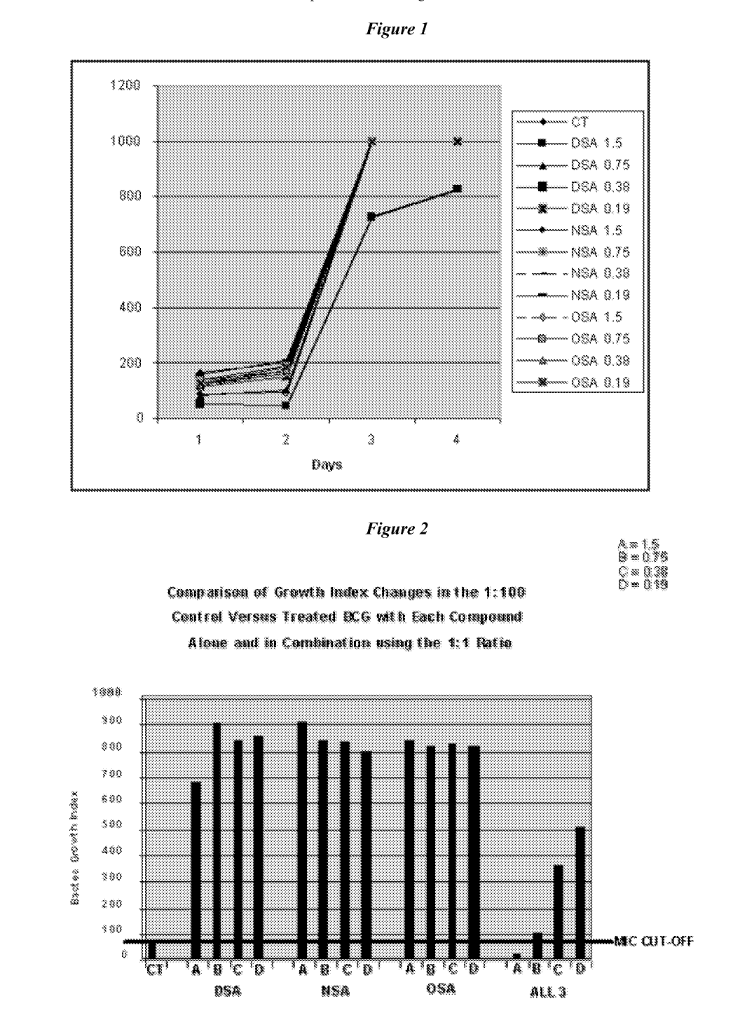

As shown in FIG. 1, only the use of DSA at 1.5 μg/ml was observed to inhibit the growth of M. bovis BCG cells. The use of DSA at 1/2, 1/4, or 1/8 of the MIC showed no effect. Similarly, the use of NSA or OSA at the same concentrations as DSA showed no effect. But a combination of DSA, NSA, and OSA, at concentrations where NSA and OSA are ineffective, was surprisingly found to be effective at inhibiting cell growth.

As shown in FIG. 2, a combination of DSA at its MIC of 1.5 μg/ml, with NSA and OSA at the same concentration, demonstrated better inhibition than DSA alone at that concentration, despite the observation that NSA and OSA alone at those concentrations were ineffective. The unexpected effectiveness of the combination was also observed as the amount of each of the three agents was reduced to 0.75, 0.375, 0.1875 μg/ml, or 1/2, 1/4, and 1/8 of each compound's MIC, respectively, as shown in FIG. 2.

While the unexpected results may be considered to be illustrated by use of DSA at its MIC and below in an in vitro assay, the phenomenon observed through the results is expected to remain regardless of the amount of DSA used so long as it is in combination with NSA and OSA in a combination which inhibits mycobacterial cell growth and/or is antimicrobial or microbicidal to the cells. Thus use of a DSA at amounts higher than its MIC in combination with NSA and OSA, at their MICs, sub-MICs, or above MICs, are also expected to produce the same phenomenon. In some embodiments, the invention provides for compositions and methods of using such combinations.

The illustrated unexpected results may also be considered from an “NSA-centric” view as based on a combination of NSA at its MIC or below in combination with DSA and OSA, at their MICs or lower. Thus in some embodiments, the invention provides combinations comprising NSA, at its MIC, sub-MIC, or above MIC, in combination with DSA and OSA in any effective amounts for the combination. Of course the results may further be considered from an “OSA-centric” point of view as based on a combination of OSA at its MIC or below in combination with DSA and NSA at their MICs or lower. Thus in other embodiments, the invention provides for combinations comprising OSA, at its MIC, sub-MIC, or above MIC, in combination with DSA and NSA in any effective amounts for the combination.

The invention may also be considered to provide means for improving the effectiveness of DSA, NSA, or OSA, whether at MIC, sub-MIC, or above MIC levels, by including the other two agents. The foregoing, while presented in terms of the sulfone forms of DSA, NSA, and OSA, are also expected to apply to the sulfoxide forms of the compounds given the level of structural similarity and the very high likelihood of the same mechanism of action.

Thus in a first aspect, the invention provides a composition comprising octanesulphonylacetamide (OSA) or the sulfoxide form thereof, nonanesulphonylacetamide (NSA) or the sulfoxide form thereof, and decanesulphonylacetamide (DSA) or the sulfoxide form thereof. In light of the observations represented by FIGS. 1-3, the amounts of each agent in a combination may vary greatly. In some embodiments, the amounts may be such that there is a definite amount of a first agent, such as, but not limited to, DSA, with sufficient amounts of the other two agents to produce an anti-mycobacterial effect greater than that of the first agent alone added to the effects of each of the other two agents. Non-limiting examples include compositions comprising DSA, in amounts of about 1, about 2, about 5, about 10, about 20, about 30, about 50, about 75, about 100, about 200, about 300, about 400, about 500, about 600, about 700, about 800, about 900, about 1000, about 1500, about 2000, about 2500, about 3000, about 3500, about 4000, about 4500, or about 5000 μg, with NSA and OSA in amounts sufficient to produce an anti-mycobacterial effect greater than that of the DSA alone added to the effects of the NSA amount alone and the OSA amount alone.

The activity of agents and compositions of the invention may be evaluated by use of an assay like that described in U.S. Pat. No. 5,614,551, which is hereby incorporated by reference as if fully set forth. The patent describes an in vitro therapeutic index based on comparison of the concentration which inhibits growth of fibroblasts to the MIC against mycobacteria for an agent. The therapeutic index is the ratio of the concentration which affects a subject's non-mycobacterial cells to the concentration which affects the target mycobacterial cells. For example, the therapeutic index may be determined by comparing growth inhibition of confluent normal fibroblasts to the dose of an agent or composition resulting in the MIC for a given mycobacterium. This MIC dose can then be tested upon confluent cultures of normal human fibroblasts to determine a therapeutic index. In some embodiments, agents or compositions of the invention will have an in vitro therapeutic index in such an assay of at least about 2, at least about 5, or at least about 10 or more.

Agents and compositions may also be characterized by the concentration required to inhibit cell growth by 50% (IC₅₀ or ID₅₀). Agents and compositions with high therapeutic index will, for example, be growth inhibitory to the mycobacterial cells at a lower concentration (as measured by IC₅₀) than the IC₅₀ for the non-mycobacterial cells. In some embodiments, compositions, the effects of which on these two cell types show greater differences, are selected for use. In further embodiments, a composition will have IC₅₀ for mycobacterial cells that is at least 1/2 log lower, or at least 1 log lower, than the composition's IC₅₀ determined for non-mycobacterial cells.

The anti-mycobacterial effect may be expressed also in terms of inhibitory or bactericidal activity in comparison to a control sample untreated with an agent or a combination of agents. Anti-mycobacterial activity which inhibits or kills at least about 50, at least about 75, at least about 80, at least about 85, at least about 90, at least about 95, at least about 96, at least about 97, at least about 98, at least about 99, at least about 99.9, at least about 99.99%, at least about 99.999%, at least about 99.9999%, at least about 99.99999%, at least about 99.999999%, or at least about 99.9999999% or more of the cells relative to control may be used as an activity level of the invention. Stated in terms of log kill, survival of less than about 10, less than about 1, less than about 0.1, less than about 0.01% of treated cells relative to control may be used as an activity level of the invention. In in vivo embodiments, the invention may be used to inhibit or kill about 10² colony forming units (CFUs) of mycobacteria as found in a ml of bodily fluid, about 10³ CFU/ml, about 10⁴ CFU/ml, about 10⁵ CFU/ml, about 10⁶ CFU/ml, about 107 CFU/ml, about 10⁸ CFU/ml, or about 10⁹ CFU/ml or higher. An activity level may also be selected based upon a time period. Non-limiting examples include level of inhibition or bactericidal after about 4, about 5, about 6, about 7, about 8, about 9, or about 10 days or more.

In other non-limiting examples, the amounts of NSA used in a composition will be equal to or greater than the amount of DSA on a weight or molarity basis. In further non-limiting examples, the amounts of OSA in a composition will be equal to or greater than the amount of NSA on a weight or molarity basis. In particular embodiments, the amounts of each agent will be approximately equal on a weight basis in a composition of the invention. In other embodiments, the amount of each agent may vary individually from each of the other two by about 1, about 2, about 3, about 4, about 5, about 6, about 7, about 8, about 9, about 10, about 11, about 12, about 13, about 14, about 15, about 16, about 17, about 18, about 19, or about 20% on a weight or molarity basis. Thus non-limiting examples of ratios of the DSA, NSA, and OSA in a composition, by weight or molarity, include 1:1:1, 1:0.9:1, 1:0.9:0.9, 1:0.8:1, 1:0.8:0.9, 1:0.8:0.8, 1:1.1:1, 1:1.1:1.1, 1:1.2:1, 1:1.2:1.1, and 1:1.2:1.2.

A composition of the invention may be in any convenient solid, liquid, or suspended form, optionally in a sterile form. In some embodiments, the composition is a pharmaceutical composition containing the agents in amounts for administration by parenteral (subcutaneously, intramuscularly, intramedullary injections, intravenously, intraoperitoneally, intrapleurally, intravesicularly, or intrathecally), intravenous application, topical (including by direct injection into a tissue or location or intravascular injection into vessels infiltrating a tissue, including intraventricular, intranasal, and intraocular), buccal, sublingual, oral, transdermal, rectal, vaginal, transmucosal, intestinal, nasal, inhalation, or by intracavity or peristaltic administration, as selected by a skilled person or necessitated by the nature or location of the disease.

For injection, the compositions of the invention may be formulated in aqueous solutions, for example, in physiologically compatible buffers such as Hank's solution, Ringer's solution, or physiological saline buffer. For transmucosal administration, penetrants appropriate to the barrier to be permeated are used in the formulation. Such penetrants are generally known in the art. For oral administration, the composition may be formulated as tablets, dragees, pills, capsules, liquids, gels, syrups, slurries, suspensions and the like.

In other embodiments, the pharmaceutical composition is suitable for non-invasive administration such as, but not limited to, (1) topical application to the skin in a formulation, such as an ointment or cream, which will retain the composition and/or agents in a localized area; (2) oral administration; (3) nasal administration as an aerosol; (4) intravaginal application of the inhibitor formulated in a suppository, cream or foam; (5) rectal administration via suppository, irrigation or other suitable means; (6) bladder irrigation; and (7) administration of aerosolized formulation of the inhibitor to the lung. Aerosolization may be accomplished by well known means, such as the means described in WO 93/12756, pages 30-32, incorporated herein by reference.

A composition of the invention may also be administered locally or topically in gels, ointments, solutions, impregnated bandages, liposomes, or biodegradable microcapsules. Compositions or dosage forms for topical application may include solutions, lotions, ointments, creams, gels, suppositories, sprays, aerosols, suspensions, dusting powder, impregnated bandages and dressings, liposomes, biodegradable polymers, and artificial skin.

A further non-limiting example of a form of the compositions is as a solid. In some cases, the solid is used as a dosage form to deliver the composition to a subject based upon amount per body mass of the subject or amount to achieve a serum or target tissue concentration in the subject. Non-limiting examples of such forms include those containing about 10, about 25, about 50, about 75, about 100, about 200, about 300, about 400, about 500, about 600, about 700, about 800, about 900, about 1000, about 1100, or about 1200 mg or more of the three agents in total per kilogram of the subject to be treated. No upper limit beyond that resulting from insolubility is yet contemplated.

Another non-limiting form is a vesicle, such as liposomes and micelles. Liposome or micelle forms of a composition of the invention may be prepared by any of the methods known in the art for preparation of liposomes and micelles containing small molecule inclusions. Liposomes that are particularly suited for aerosol application to the lungs are described in WO 93/12756, pages 25-29, incorporated herein by reference. Vesicles such as liposomes and micelles may be optionally dried, such as by freeze drying or other functionally equivalent methods to produce a dried form. The dried form may be optionally manipulated as a solid, such as to prepare a powder, and rehydrated to reconsititute the vesicles before use. A powder form may alternatively be administered to a subject such that it is reconstituted into the vesicles by hydration in vivo.

A composition of the invention may further containing additional agents, such as, but not limited to, one or more other anti-mycobacterial, antibiotic, antifungal or antiviral substance, including one or more additional agents suitable for inhibiting mycobacteria or treating a mycobacterial infection. Non-limiting examples of other anti-mycobacterial agents include those used for treating tuberculosis (e.g. long-acting rifamycins, fluoroquinolones, oxazolidinones, nitroimidazoles, diarylquinolines, rifampin, isoniazid and pyrazinamide). A composition may also be used in a method in combination with one or more non-chemical method used to inhibit mycobacteria or treat a mycobacterial infection. Of course the invention also provides for a method of using a composition of the invention in combination with one or more additional agents suitable for inhibiting mycobacteria or treating a mycobacterial infection.

In other embodiments, the additional agent is a carrier for a composition of the invention. The carrier may be a pharmaceutically acceptable carrier or excipient, optionally containing other components so long as the other components do not reduce the effectiveness of the DSA, NSA, and OSA agents in the composition so much that their activity is negated. Pharmaceutically acceptable carriers typically include carriers known to those of skill in the art, including pharmaceutical adjuvants. Generally pharmaceutically acceptable carriers include water, saline, Ringers solution, Ringer's lactate, 5% dextrose, buffers, and other compounds described, e. g., in the MERCK INDEX, Merck & Co., Rahway, N.J. See also, Gilman et al. (eds) (1990) Goodman and Gilman's: The Pharmacological Bases of Therapeutics, 8th Ed., Pergamon Press; Novel Drug Delivery Systems, 2nd Ed., Norris (ed.) Marcel Dekker Inc. (1989); Remington's Pharmaceutical Sciences, Mack Publishing Co., Easton, Pa., 1985; and Bioreversible Carriers in Drug Design, Theory and Application, Roche (ed.), Pergamon Press, (1987). In some embodiments, the carrier may be a lipoprotein suitable for use in the composition. A composition may also contain one or more auxiliaries which facilitate processing of the composition into preparations which can be used pharmaceutically.

Non-limiting examples of pharmaceutical carriers include alginates, carboxymethylcellulose, methylcellulose, agarose, pectins, gelatins, collagen, vegetable oils, peanut butter, mineral oils, stearic acid, stearyl alcohol, petrolatum, polyethylene glycol, polysorbate, polylactate, polyglycolate, polyanhydrides, phospholipids, polyvinylpyrrolidone, and the like. The compositions may also be formulated for delayed release (e.g. slowed release over time) or alternatively for rapid release. Preparations for oral use can be prepared by combining a composition of the invention with a solid excipient, optionally grinding the resulting mixture, and processing the mixture of granules, after adding suitable auxiliaries, if desired, to obtain tablets or dragee cores. Suitable excipients include, but are not limited to, fillers such as sugars, including lactose, sucrose, mannitol, or sorbitol; and cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). In some embodiments, a disintegrating agent may be added, such as the cross-linked polyvinylpyrrolidone, agar, or alginic acid or a salt thereof such as sodium alginate.

When used, dragee cores are provided with a suitable coating. In some embodiments, a concentrated sugar solution may be used, optionally containing gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of agents in the composition.

Pharmaceutical preparations that can be used orally further include push-fit capsules made of gelatin, as well as soft, sealed capsules made of gelatin, and a plasticizer, such as glycerol or sorbitol. The push-fit capsules may contain a composition of the invention in admixture with a filler such as lactose, a binder such as starch, and/or a lubricant such as talc or magnesium stearate. Optionally, a stabilizer may be included. In soft capsules, the agents of the composition may be dissolved or suspended in suitable liquids, such as fatty oils, liquid paraffin, or liquid polyethylene glycols (PEGs), optionally in the presence of a stabilizer.

The invention further provides a mycobacterial cell contacted with a composition described herein. In some embodiments, the mycobacterial cell is a pathogenic cell, which may be considered to be those capable of causing a communicable disease. In other embodiments, the mycobacterial cell may be one which is sensitive or susceptible to one or more of DSA, NSA, or OSA, or the sulfoxide forms thereof. Sensitivity or susceptibility may be by use of any suitable assay, including growth inhibition as described herein. Alternative assays include those for evaluating decreases in ATP levels or other inhibition of energy metabolism. Sensitivity or susceptibility of drug resistant or multi-drug resistant mycobacterial cells may also be used. In other embodiments of the invention, a sensitive or susceptible mycobacterial cell may be one which is not considered pathogenic. A non-limiting example is a mycobacterium which can give rise to an opportunistic infection.

In additional embodiments, the mycobacterial cell may be actively growing, such as in a case of acute infection in vivo. In other embodiments, the mycobacterial cell is quiescent, or otherwise not actively growing, such as in a case of a latent infection in vivo.

In further embodiments, use of a composition to contact a mycobacterial cell inhibits growth or proliferation of the cell. Without being bound by theory, and offered based on in vitro observations to improve the understanding of the invention, the inhibition is based on bactericidal rather than bacteriostatic activity. The inhibited cell may be a pathogenic mycobacterial cell, whether actively growing or latent, as described herein. The inhibitory activity may occur over time, or after passage of a period of time.

The invention also provides methods to inhibit growth or proliferation of a mycobacterial cell. One method comprises contacting a mycobacterial cell with a composition of the invention. The amount of the composition should be an effective amount or effective dose to achieve a level or inhibition and/or bactericidal activity. The contacted cell, such as a pathogenic mycobacterium, is inhibited by the composition. The term “effective amount” or “effective dose” as used herein typically refers to the amount of the composition of the invention, which is required to achieve the desired activity or result. As a non-limiting example in therapeutic applications, an effective amount is the amount required to be administered to a patient to result in treatment of the condition, such as mycobacterial infection, for which treatment is needed or sought. Thus the amount may be that which inhibits mycobacteria to a particular level or degree, or which treats the infection, and/or any related symptoms, to a particular level or degree. In terms of prophylactic applications, an effective amount is that required to be administered to a subject to result in a state of protection, inhibition of infection, or inhibition of an infection's progress, to a particular level or degree. Effective amounts may be readily determined by the skilled person depending upon factors such as the nature of the infection, the type of mycobacteria, the mode of administration, and the size and health of the patient.

In other embodiments, the invention provides for use of a composition comprising the three agents wherein one or more of them is/are present in the form of a pro-drug which is converted to the active agent after application. Depending on the location of the mycobacteria, the conversion may occur intracellularly or otherwise in vivo, such as in a subject. Alternatively, the composition may comprise the three agents wherein one or more of them is/are present in the form of an active metabolite as would occur after administration in vivo.

With respect to pro-drugs, their therapeutic utility is widely recognized by skilled persons. A biologically active molecule is chemically linked to moieties that alter the pharmacokinetic properties of the molecule to form a pro-drug thereof. As a non-limiting example, pro-drugs have been designed and synthesized that enhance absorption from the gastrointestinal tract, resist chemical breakdown in the acidic pH of the stomach, improve tissue distribution, release the therapeutic molecule at the primary site of action, modulate plasma clearance, increase solubility, prolong the release time of an agent, and the like. Overall, and in-vivo, the pro-drug breaks down to release the biologically active molecule and the relatively inert linked moiety.

Representative pro-drugs of two classes were synthesized to modify the pharmacokinetic properties of DSA. One class may be termed N-acyloxymethyl pro-drugs and is represented by the following formula:

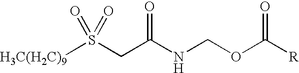

The methyl ester (where R═CH₃) was synthesized as a representative of this class. It was expected to hydrolyze quickly in the stomach, be absorbed rapidly and distribute widely through tissues. The MIC of this compound against M.tb was 6.25 μg/ml compared to an MIC of ˜1.0 μg/ml for DSA.

A second class of pro-drugs was synthesized using N-Mannich bases. Two representative compounds of this class were made, one with an 8 carbon aliphatic side chain and the second with an aliphatic side chain 10 carbons in length. The second is represented by the following formula:

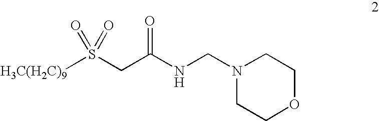

This compound resists decomposition in acid pH and is expected to be absorbed as the intact pro-drug molecule. At physiologic pH (or about 7.5), the molecule slowly breaks down (with a half-time of about 15 hours) to release DSA. This property would enhance the prolonged maintenance of plasma concentrations of the active drug. The MICs of these pro-drug molecules are 6.25 μg/ml (for the 10 carbon compound) and 12.5 μg/ml (for the 8 carbon compound).

The compositions of the invention may comprise one or more of these pro-drugs as well as analogous pro-drug forms of NSA and OSA having the same linked chemical moiety.

The inhibition of mycobacteria may occur in the context of an animal subject. In some embodiments, the animal is a domesticated (e.g. livestock) or otherwise subject to human care and/or maintenance (e.g. zoo animals and other animals for exhibition). In other embodiments, the animal is feral or otherwise in its natural environment. The animals may be simply carriers of mycobacteria or afflicted with an active infection. Non-limiting examples of animals include both ruminants and carnivores, such as dogs, cats, birds, horses, cattle, sheep, goats, marine animals and mammals, penguins, deer, elk, foxes, and prairie dogs. Potential use in livestock or other veterinary applications include treatment of infections by M. paratuberculosis, also known as Johne's bacillus, an organism that produces a chronic enteritis in ruminants (e.g., cattle and sheep) and which is invariably fatal.

The inhibition may also be in a human subject, such as a patient seeking medical treatment or care. The human subject may be one afflicted with an active mycobacterial infection or carrier of latent mycobacteria. With respect to the latter, the compositions of the invention can aid in treating millions of potential patients who harbor quiescent disease which may become active as a result of immunosuppression or other systemic disease. A non-limiting example of immunosuppression is HIV infection or AIDS. In immunocompromised or immunosuppressed subjects, a composition may also be used against “atypical” or “non-tuberculosis mycobacteria” such as M. avium intracellulare, a common AIDS pathogen, and other species that are commonly drug resistant. Moreover, and given the biochemical similarity between M. tuberculosis and M. leprae, the compositions of the invention may be used in the treatment of leprosy (Hansen's disease). Treatment of human patients infected with M. paratuberculosis, as well as Crohn's Disease, is also provided by the invention.

The animal and/or human subject to be treated may be one which has been diagnosed as having mycobacteria or a disease caused by a mycobacterium and thus in need of treatment. As used herein, infection refers to the presence and/or multiplication of a microorganism within or on a host subject's body. An infection which disrupts the normal function(s) of the host subject results in disease caused by the infection. The subject may also be determined to be in need of treatment to prevent the spread of mycobacterial infection to other subjects. Non-limiting examples of infection for which treatment may be warranted include pulmonary infection, symptomatic infection, asymptomatic infection, subclinical infection, inapparent infection, opportunistic infection, local infection, systemic infection, focal infection, primary infection, secondary infection, mixed infection, acute infection, chronic infection, subacute infection, latent infection, infection of one or more specific tissues (e.g. lung), bacteremia, septicemia, and intracellular infection.

Selection of subjects for treatment may be made by any suitable means known in the art. In some embodiments, the detection of mycobacteria may be by culture, antigen testing (e.g. skin test), X-ray analysis, direct examination of a subject, direct nucleic acid hybridization techniques, such as PCR, or by microscopic identification of biopsies or fluids from the patient. The detection, or suspicion, of mycobacteria presence, may be used to identify a subject for treatment as described herein. In some cases, the treatment is a prophylactic treatment; in other cases, the treatment is lengthened in light of the selection findings. The invention maybe applied to the treatment of diseases which cause lesions in externally accessible surfaces of the infected animal. In some embodiments, externally accessible surfaces include all surfaces that may be reached by non-invasive means (without cutting or puncturing the skin), including the skin surface itself, mucus membranes, such as those covering nasal, oral, gastrointestinal, or urogenital surfaces, and pulmonary surfaces, such as the alveolar sacs. Of course systemic infections may also be treated. A non-limiting example is disseminated M. tuberculosis.

In other embodiments, the identification of subjects may be based on the presence of a factor that indicates exposure to mycobacteria and/or susceptibility to mycobacteria. Non-limiting examples of the former include a positive skin test and/or positive chest X-ray analysis. Non-limiting examples of the latter includes expression of high levels of the chemokine monocyte chemoattractant protein-1 (MCP-1), expression of low levels of IL-12p40, or the presence of the -2518G allele of MCP-1 (see for example Flores-Villanueva et al. J. Exp. Med. 202(12):1649-1658, 2005). Increased levels of MCP-1, and decreased levels of IL-20p40 may be determined based upon comparisons to normal subjects, such as human patients. Non-limiting examples of detection or determination means include, but not limited to, antibody mediated detection in blood or plasma as well as quantitative RT-PCR to detect mRNA that reflects MCP-1 and IL-12p40 expression. In some embodiments, a ratio of MCP-1 expression level to IL-12p40 expression level may be used as an indicator of susceptibility to mycobacteria. Detection of the -2518G allele, or other allele that increases MCP-1 expression, may be by any appropriate means including, but not limited to, PCR analysis, restriction enzyme polymorphism, nucleic acid hybridization, and detection of heterozygosity or homozygosity for the -2518G allele.

With identification of a subject as susceptible to mycobacteria, which includes susceptibility to reactivation of a latent mycobacterial infection and to an active pulmonary infection as non-limiting examples, the subject may be treated with a method as described herein. The method may be for prophylaxis to prevent an initial infection, such as in cases where the subject has no indication of having been exposed to or infected with mycobacteria, or for prophylaxis to prevent development of an acute mycobacterial infection, such as in cases of a subject that has been exposed to mycobacteria and cases of “re-activation” of latent mycobacteria to cause an acute infection. Identification of susceptibility, such as by increased expression of MCP-1, may also be used to indicate a longer treatment term with a composition of the invention for the identified subject. Non-limiting examples of a longer treatment term include a term that is about 10%, about 20%, about 30%, about 40%, about 50%, about 100%, about 150%, about 200%, about 300%, about 400%, or about 500% or more, lengthier than treatment of a patient without the susceptibility.

The treatment methods of the invention are with compositions at a level that does not kill the subject or irreversibly injure vital organs, or lead to a permanent reduction in liver function, kidney function, cardiopulmonary function, gastrointestinal function, genitourinary function, integumentary function, musculoskeletal function, or neurologic function. It is possible, however, to administer a composition at a level that causes some host cell injury or death which is subsequently regenerated (e.g., endometrial cells).

Beyond treatment of a mycobacterial infection, the invention also provides for prophylactic use of the compositions described herein. In one sense, prophylaxis is with respect to the development of an acute mycobacterial infection, whether by infection that leads directly to active disease or by “re-activation” of latent mycobacteria to cause acute infection. In a second sense, prophylaxis is with respect to preventing initial infection, and so stopping the generation of either an acute or latent infection.

Thus the invention provides for the administration of a composition described herein to prevent or reduce the likelihood of mycobacterial infection. Such a method may act by conditioning the subject to resist mycobacterial infection. The method may comprise administering a composition to a subject prior to contact with or exposure to mycobacteria. Alternatively, the administering may be to a subject already contacted by, exposed to, or infected by, mycobacteria. The amount of composition may be any effective amount or effective dose to achieve the necessary or desired effect of prevention, prophylaxis, or reduction in likelihood.

The invention further provides dosages and dose schedules for use of the described compositions. Generally, the dose may be any that achieves a desired outcome, inhibitory activity, or level of agent in a subject. But as is understood by a skilled person, dose and duration of therapy depends on a variety of factors, including, but not limited to, the therapeutic index of the composition used, mycobacteria involved, disease type, patient age, patient weight, and tolerance of toxicity. The dose will usually be chosen to achieve tissue, serum, or other bodily fluid concentration levels from about 1 ng to about 1000 g/ml, about 10 ng to about 100 g/ml, about 100 ng to about 10 g/ml, about 1 μg to about 1 μg/ml, about 10 μg to about 100 mg/ml, about 100 ng to about 10 mg/ml, or about 1 mg/ml. Non-limiting examples of bodily fluids of the invention include sputum and saliva.

In some embodiments, an initial dose level is selected based on the concentrations shown to be effective in in vitro and in vivo models and in clinical trials, up to maximum tolerated levels. The dose of a composition and duration of therapy for a particular subject can be determined by a skilled practitioner using standard pharmacological approaches in view of the above factors. Response(s) to treatment may be monitored by analysis of blood, serum or other body fluid levels of the agents in a composition of the invention, measurement of activity of an agent or its level in relevant tissue, serum or other bodily fluid, or monitoring the disease state of the subject. The skilled practitioner may then adjust the dose and duration of therapy based on the response(s).

In addition to the above, the invention provides a method to achieve a desired dose in a subject, such as a desired level of anti-mycobacterial agent in a tissue, serum or other bodily fluid, by administering a composition of the invention. The composition may be in a dose form as described above. The administering may be of the same dosage over a period of time or administering non-identical doses over time. The periodicity of the doses may be regular (e.g. daily, hourly, etc.) or irregular.

Thus the invention further provides for the use of a dosing schedule based on non-identical doses over a period of time. In some embodiments, the schedule comprises the administration of one or more larger initial dose(s) followed by one or more lower maintenance dose(s). The initial dose(s) may be based upon pharmacological considerations and to load tissues or bodily fluids with a concentration of composition or agent(s) therein in a short period of time. The subsequent maintenance dose(s) are to keep the concentration at a sustained level over a remaining period of time. The concentrations may be any suitable for achieving the desired result, whether treatment or prophylactic in nature. Non-limiting examples include concentrations that is a whole number multiple of an MIC.

In additional aspects, the invention provides methods to inhibit or reduce energy metabolism in a mycobacterial cell by contacting the cell with a composition as described herein. In some embodiments, the methods inhibit ATP synthesis and/or interfere with cellular respiration. The methods may also produce multiple downstream effects resulting from ATP synthesis inhibition and/or interference with cellular respiration. Non-limiting examples include a decrease in the energy-dependent synthesis of other macromolecules, such as proteins and mycolic acids. Thus the invention also provides a method to inhibit protein and/or mycolic acid synthesis in a mycobacterial cell by contacting said cell with a composition as described herein. The term mycolic acids refers to α-substituted, β-hydroxy fatty acids, such as of about C90 as present in the cell walls of some mycobacteria.

Such methods of using a combination of the invention may be performed in combination with one or more additional agents which inhibit mycobacteria or treat a mycobacterial infection as described herein. The likely degradation of the mycobacterial cell wall by inhibition of mycolic acid synthesis will likely result in permeability for one or more additional agents to more effectively target the cell. Non-limiting examples of an additional agent include other protein synthesis inhibitors as known to the skilled person.

The invention further provides for a method to treat a mycobacterial infection with reduced emergence of resistance in the treated mycobacteria. The method may also be considered to be treating mycobacteria with reduced or minimized selection for resistant mycobacteria. As would be understood by the skilled person, the emergence of, or selection for, resistance to a compound by an organism may occur where a population of the organism is placed under selective pressure such that members of the population unable to survive under the pressure will be eliminated from the population. Thus, the members of the population able to survive will emerge as organisms resistant to the selective pressure.

The rapid emergence of drug resistance constitutes a major limitation to the curative potential of the therapeutics used in the treatment of tuberculosis. Significant drug resistance usually emerges over a period of several days and is generally apparent early in the course of treating patients with single agents. As a non-limiting example, consider the fact that a patient presenting with acute pulmonary tuberculosis is likely to be infected with 10⁶ to 10¹² viable bacilli. As shown in the table below, treatment with a single agent will likely reduce the number of viable organisms and temporarily resolve the patient's symptoms. However, the emergence of drug resistant bacilli over a population of patients is inevitable. As a non-limiting instance, isoniazid monotherapy results in approximately 1 out of every 10,000 bacilli developing spontaneous chromosomal mutations which confer resistance. Other drugs do not fare much better when used alone.

TABLE 1
Primary rate of resistance commonly ascribed to current
first-line anti-tuberculosis drugs.

| Drug | Primary Rate of Resistance* |
|---|---|
| Ethambutol (EMB) | 1 in 10⁴ |
| Isoniazid (INH) | 1 in 10⁴ to 1 in 10⁶ |
| Streptomycin (STR) | 1 in 10⁶ |
| Rifampin (RIF) | 1 in 10⁸ |
| INH + RIF | 1 in 10¹⁴ |
| DSA | <1 in 10** |
| DSA + NSA + OSA | at least <1 in 10⁹** |
| *R. M. Jasmer M.D. CDC sponsored 4^{th} National Conference on TB. 2002 | |
| **Primary rates of resistance for M. bovis BCG against DSA and the DSA + NSA + OSA combination was determined in an in-vitro culture assay. The results reflect experiments in addition to those of Example 4 below. Organisms were grown to a density of 10⁷ to 10⁹ in the presence or absence of a sub-inhibitory concentration of each compound (down to one-quarter the respective MIC's for each compound). Cultures were subsequently incubated for 7 days | |
| # and any resistant colonies counted. | |

Therefore, and to avoid the emergence of resistance, current regimens require that combinations of drugs be used to treat active disease. This practice decreases the rate at which resistance emerges as illustrated by the combination of INH and RIF in the table above. Also as seen in the table, the primary rate of resistance seen with DSA is very low and compares favorably to other drugs. Resistance to the DSA+NSA+OSA combination emerges even slower and the primary rates of resistance appear to be equal to or lower than rates associated with currently used combinations of drugs.

The emergence of resistance with some bactericidal compounds may occur because the selective pressure is insufficient to leave most members of the population unable to survive. In some cases, this occurs because the amount of the bactericidal compound used, and so the bactericidal effect, is insufficient to be lethal to enough members of the population. The present invention provides for the use of the disclosed compositions in amounts, or at levels, at which the emergence of resistance is at a frequency of less than 1 in 10⁷, less than about 1 in 10⁸, less than about 1 in 10⁹, or less than about 1 in 10¹⁰ mycobacteria contacted with the composition. The frequency may be determined based upon the detection of colony forming units (CFUs) versus the density of organisms in vitro. This compares very favorably with the frequency of resistance with the use of DSA, NSA, and OSA individually, where resistance occurs at a frequency of 1 in 10⁷ mycobacteria.

Reducing or minimizing resistance is advantageously used in an in vivo context as disclosed herein because the counterpart to a CFU within a mycobacteria infected host is the emergence of a resistant infection. Thus the methods of the invention may be advantageously used to reduce or minimize the occurrence of resistant mycobacteria in an infected host by treatment with a composition of the invention.

Based on the above, the invention also provides a method to present selective pressure on a population of mycobacteria by contacting the population with a composition of the invention to apply the selective pressure. The selective pressure may be to inhibit or reduce mycobacterial growth, to kill the mycobacterial cells, and/or to inhibit or reduce energy metabolism in the mycobacterial cells with decreased occurrence of resistance to the composition. A method may comprise applying of the selective pressure, comprising the contacting of mycobacteria with an effective amount of a composition of the invention, so that emergence of resistance to the composition is reduced when compared to use of the individual agents of the composition.

A composition of the invention may be prepared by combining individual components. In embodiments of the former, the composition is prepared by combining more than one preparation, wherein each preparation comprises at least one of DSA, NSA, and OSA, or the sulfoxide forms thereof. Each preparation comprising at least one of DSA, NSA, and OSA, or the sulfoxide forms thereof, may be produced by any suitable means known to the skilled person. Such preparations may optionally already contain an additional component for inclusion in the final composition as described herein.

Having now generally described the invention, the same will be more readily understood through reference to the following examples which are provided by way of illustration, and are not intended to be limiting of the present invention, unless specified.

### EXAMPLES

### Example 1

### Susceptibility Testing and MIC Determination

Susceptibility testing and MIC determination for each compound against M. tuberculosis (H37Rv) were performed using the standard BACTEC radiometric growth system (Becton Dickinson, Sparks, Md.). This was followed by subsequent testing of additional mycobacterial species such as M. bovis BCG with selected compounds using the same standard procedure. Initial stock solutions (1 mg/ml) and subsequent dilutions of experimental compounds were prepared in DMSO (Sigma). Stock concentrations of each inhibitor (0.1 ml) were then added to individual 4.0 ml BACTEC bottles resulting in the following final concentrations (μg/ml): 50, 25, 12.5, 6.25, 3.0, 1.5.

For each strain tested a 1.0 McFarland suspension was prepared and 0.1 ml was added to each of the following bottles: a direct control (bottle containing diluent, DMSO, but no antibiotic), a control containing a 1:100 organism dilution (also without antibiotic) and each concentration of drug. All bottles were incubated at 37° C., and the growth index (GI) of each bottle was recorded daily until the GI of the 1:100 control reached 30. The minimum inhibitory concentration (MIC) of each isolate was determined using the following criterion: once the growth index (GI) of the 1:100 control bottle had reached a value of 30 the growth index change (Δ) was calculated for a one day period at each concentration tested.

The MIC was defined as the lowest inhibitor concentration that yielded a growth index change less than that of the 1:100 control bottle. A modification of this protocol was used for MIC determinations of MAC and M. avium subspecies paratuberculosis (Parrish, 2004). Susceptibilities and MIC determinations of M. tuberculosis isolates to isoniazid, streptomycin, ethambutol, rifampin, and pyrazinamide were done using standard BACTEC methods (Siddiqi, 1992). All primary drugs were purchased from Becton Dickinson (Sparks, Md.).

### Example 2

### Inhibition of Mycobacterium bovis BCG

The inhibition assay was performed essentially as described in Example 1. The modification was that in combination studies, 0.1 ml of each compound was added to each test vial to achieve the final desired concentration. Since this resulted in a total volume of 0.3 ml being added to each test vial (for the triple combination), a separate “dilution control” was added in which 0.3 ml of DMSO (diluent for all compounds) to ensure that no confounders existed due to the increased volume in the test vials.

Two different ratios were tested in combination studies. Each of DSA, NSA, and OSA was combined at their respective MIC (DSA 1.5 μg/ml, NSA 3.0 μg/ml, and OSA 6.25 μg/ml) followed by a two-fold dilution series maintaining this particular ratio of one agent to another (approximately a 6:3:1 ratio by weight).

A second ratio was tested in which each agent was combined at the same concentration starting at 1.5 μg/ml and then followed by two-fold dilutions down to 0.1875 μg/ml (while maintaining a 1:1:1 ratio by weight).

The following Table 2 illustrates the amounts used. All concentrations are in μg/ml.

TABLE 2

| Ratio | DSA | NSA | OSA |
|---|---|---|---|
| 6:3:1 | | | |
| A | 1.5 | 3.0 | 6.25 |
| B | 0.75 | 1.5 | 3.125 |
| C | 0.375 | 0.75 | 1.5625 |
| D | 0.1875 | 0.375 | 0.78125 |
| 1:1:1 | | | |
| 1.5 | 1.5 | 1.5 | 1.5 |
| 0.75 | 0.75 | 0.75 | 0.75 |
| 0.38 | 0.375 | 0.375 | 0.375 |
| 0.19 | 0.1875 | 0.1875 | 0.1875 |

The results are shown in FIG. 3.

### Example 3

### Inhibition of PTB (paratuberculosis)

The inhibition assay was performed essentially as described in Example 1 for TB and BCG. Briefly, PTB inoculum was standardized by use of “seed vials” of a suspension of microorganisms in supplemented media. The suspension was vortexed with glass beads and then 0.3 ml to 0.5 ml was introduced into individual supplemented BACTEC 12B vials. Vials were subsequently incubated at 37° C. and the GI recorded until reaching 999. At that time, 0.3 ml of the “seed vial” was introduced into test and compound containing vials. Vials were then read at 24-hour intervals and the rest of the procedure followed as for TB and BCG. The MIC determination was the same as for TB and BCG.

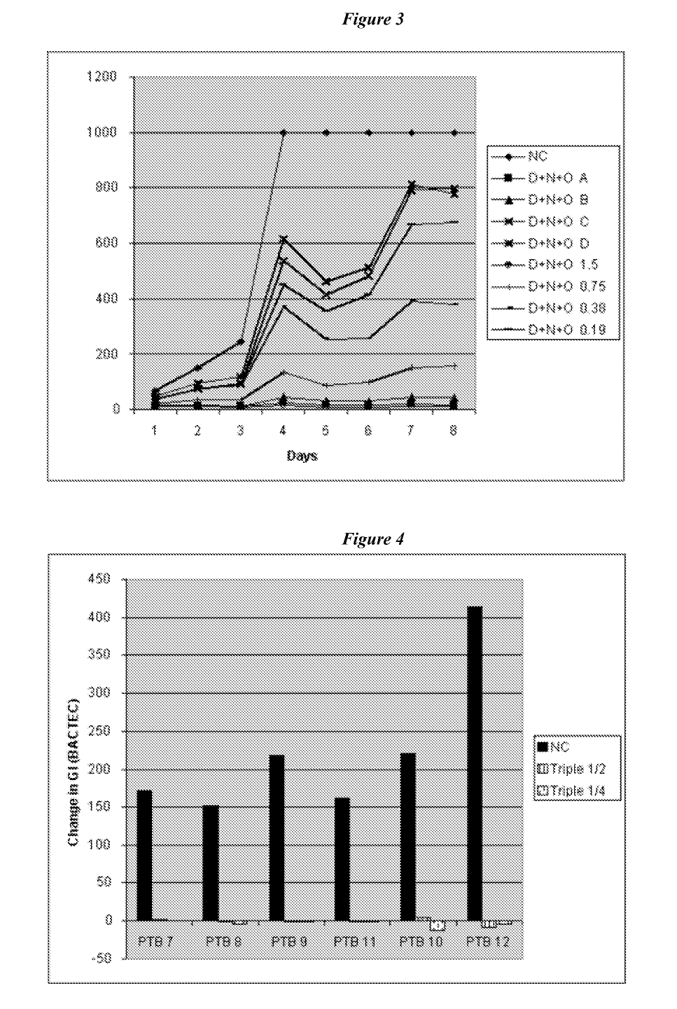

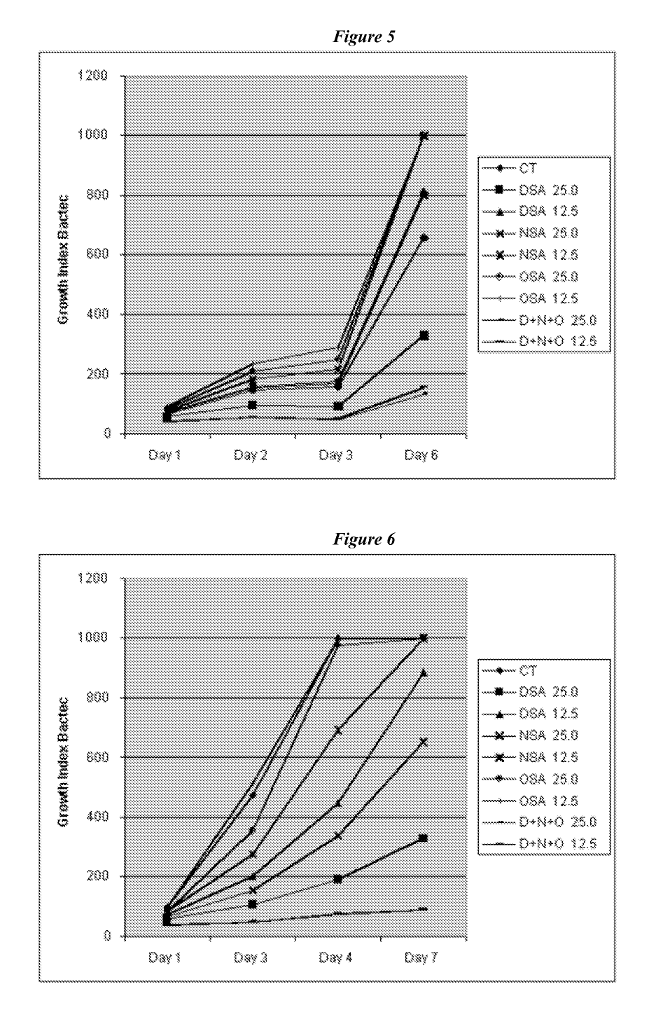

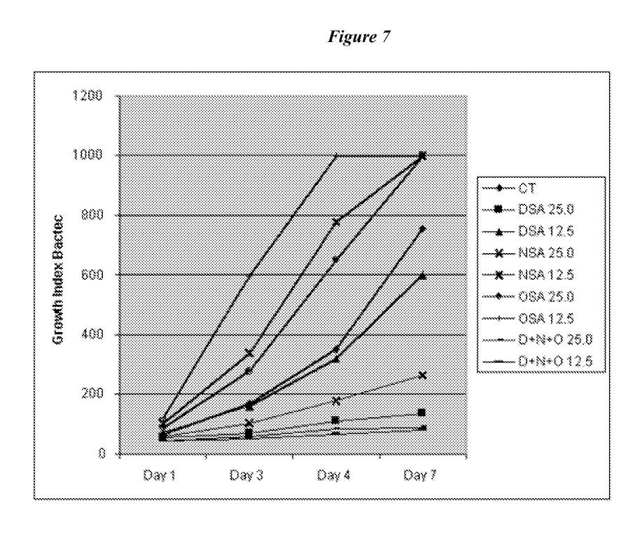

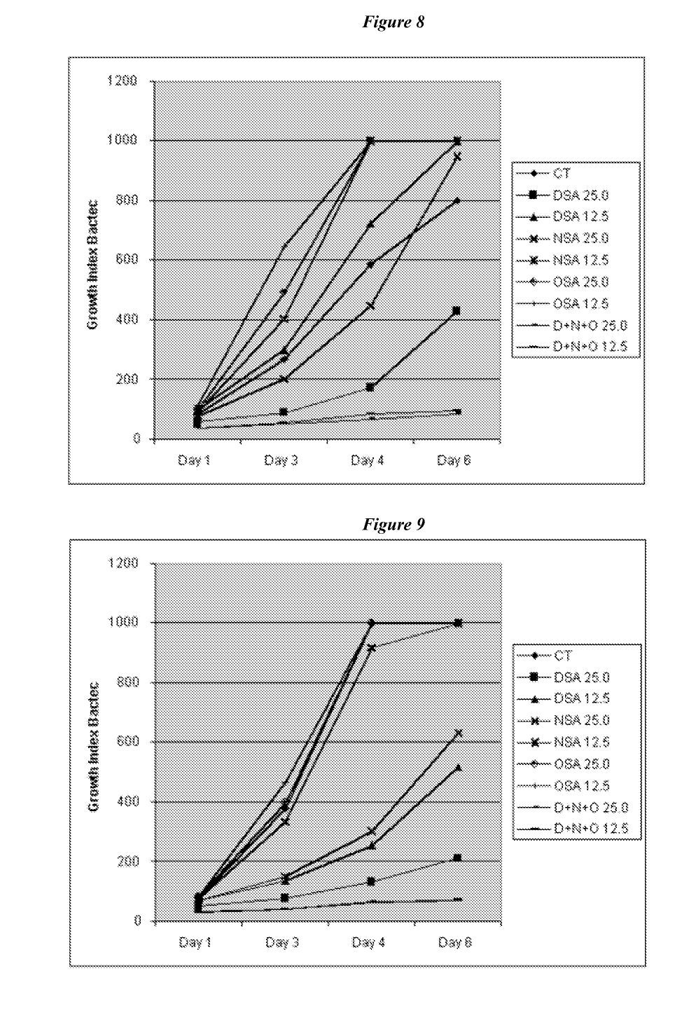

With reference to FIGS. 4-9, various strains of PTB were tested and found to have MICs of 25 μg/ml or higher with DSA, the most active of the three agents. MICs for NSA and OSA are considerably higher. MICs for some strains could not be accurately tested because they are beyond the solubility limit of the agents. Both primary and cultured PTBs were tested

All tested PTB primary strains were susceptible to combinations of DSA, NSA, and OSA when mixed in a 1:1:1 by weight ratio.

### Example 4

### Emergence of Resistance and Lack of Resistance

Different concentrations of DSA, NSA, and a combination of DSA, NSA, and OSA were used to detect the emergence of resistant mycobacteria in an in vitro assay. Cultures of mycobacteria at densities of 10⁶ and 10⁷ were used in duplicate and contacted with DSA and NSA at their respective MICs. Cultures at a density of 10⁷ were contacted with a combination of DSA, NSA, and OSA (in an approximately 6:3:1 ratio by weight) at their respective MICs, at ½ of their respective MICs, and at ¼ of their respective MICs. Resistance was detected on 25.0 μg/ml agar plates.

The results are show in Table 3 below.

TABLE 3

| | | | Number of | | |
|---|---|---|---|---|---|
| | Concentration | | resistant | Density | |
| Exposure | (μg/ml) | Days | organisms | of culture | Replicates |
| None | | N/A | 0 | 10⁷ | 5 |
| DSA | MIC 1.5 | 7 | 1 | 10⁷ | 2 |
| NSA | MIC 3.0 | 7 | 1 | 10⁷ | 2 |
| DSA | MIC 1.5 | 7 | 0 | 10⁶ | 2 |
| NSA | MIC 3.0 | 7 | 0 | 10⁶ | 2 |
| D + N + O | MIC | 7 | 0 | 10⁷ | 2 |
| D + N + O | ½ MIC | 7 | 0 | 10⁷ | 2 |
| D + N + O | ¼ MIC | 7 | 0 | 10⁷ | 2 |

Table 3 shows that DSA and NSA, at their MICs of 1.5 and 3.0 μg/ml, respectively) result in the appearance of one resistant CFU per 10⁷, but not 10⁶, cells. But a combination of DSA, NSA, and OSA resulted in no resistance even with 10⁷ cells and ¼ of the MICs for each of the agents. Resistance to OSA emerges at a frequency of less than one in 10⁶. All resistant isolates recovered were resistant at concentrations up to the limit of solubility (50 μg/ml) for each agent.

All references cited herein, including patents, patent applications, and publications, are hereby incorporated by reference in their entireties, whether previously specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A composition of matter comprising octanesulphonylacetamide (OSA) or the sulfoxide form thereof, nonanesulphonylacetamide (NSA) or the sulfoxide form thereof, and decanesulphonylacetamide (DSA) or the sulfoxide form thereof, wherein OSA, NSA, and DSA are represented by the formula
NH₂—CO—CH₂—SO₂—(CH₂)ₙ—CH₃,
wherein n is 7, 8, and 9.

2. The composition of claim 1 wherein DSA as represented by the formula
NH₂—CO—CH₂—SO₂—(CH₂)₉—CH₃,
or the sulfoxide form thereof,
is present at a concentration of about 1, or about 1.5 μg/ml or higher.

3. The composition of claim 1 wherein DSA as represented by the formula
NH₂—CO—CH₂—SO₂—(CH₂)₉—CH₃,
or the sulfoxide form thereof,
and one of the other two molecules, or the sulfoxide forms thereof, is present in approximately equal amounts by weight.

4. The composition of claim 3 wherein OSA, NSA, and DSA, or the sulfoxide forms thereof, are present in approximately equal amounts by weight.

5. The composition of claim 1 wherein OSA, NSA, and DSA are all sulfones or
only one or two of OSA, NSA, and DSA are in their sulfoxide forms.

6. The composition of claim 1, wherein said composition is in the form of a vesicle, such as a liposome or micelle, or
in a dried form suitable for rehydration to form vesicles, such as liposomes or micelles.

7. The composition of claim 1, wherein said composition is a solid dosage formulation to provide at least about 1 mg of said composition per kg of a subject.

8. The composition of claim 1, wherein said composition further comprises a carrier, a pharmaceutically acceptable excipient, or a solubility enhancer.

9. A mycobacterial cell contacted with a composition of claim 1.

10. The cell of claim 9, wherein said mycobacterial cell is pathogenic.

11. The cell of claim 10, wherein said mycobacterial cell is selected from Mycobacterium tuberculosis, drug resistant Mycobacterium tuberculosis, Mycobacterium bovis, Mycobacterium bovis BCG, Mycobacterium kansasii, Mycobacterium avium, Mycobacterium avium intracellulare, Mycobacterium leprae, Mycobacterium ulcerans, and Mycobacterium paratuberculosis.

12. A method to inhibit growth or proliferation of a mycobacterial cell, said method comprising contacting a mycobacterial cell with a composition of claim 1.

13. The method of claim 12 wherein said cell is in an animal subject.

14. A method of treating infection by a mycobacterial cell, said method comprising administering a composition of claim 1 to a subject infected with a mycobacterial cell.

15. The method of claim 12 wherein said mycobacterial cell is a pathogenic mycobacterial cell.

16. The method of claim 12 wherein said subject is a human being.

17. The method of claim 12 wherein said composition is bactericidal to said mycobacterial cell or results in a cell wall decrease in said cell.

18. The method of claim 15 wherein said mycobacterial cell is selected from Mycobacterium tuberculosis; Mycobacterium bovis, Mycobacterium bovis BCG (Bacillus Calmette-Guerin), Mycobacterium kansasii, Mycobacterium avium, Mycobacterium avium intracellulare, Mycobacterium leprae, Mycobacterium ulcerans, and Mycobacterium avium subspecies paratuberculosis.

19. A method to inhibit energy metabolism of a mycobacterial cell, said method comprising contacting said mycobacterial cell with a composition of claim 1.

20. A method to prepare a composition of claim 1, said method comprising combining more than one preparation, wherein each preparation comprises at least one of DSA, NSA, and OSA, or the sulfoxide forms thereof.
